(19) United States
(12) Patent Application Publication
(10) Pub. No.: US 2026/0262637 A1
(43) Pub. Date: Sep. 10, 2026

(54) COMPOSITIONS CONTAINING PEROXYACIDS TO CONTROL PATHOGEN GROWTH IN ANIMAL FEED AND RELATED METHODS

(71) Applicant: KEMIN INDUSTRIES, INC., DES MOINES, IA (US)

(72) Inventors: Jatuporn SALAKLANG, Clive, IA (US); Agnes Hwee Hong THNG, Hougang (SG); Haridasan CHIRAKKAL, Herentals (BE); Michael Robert Stang, Clive, IA (US)

(73) Assignee: KEMIN INDUSTRIES, INC., DES MOINES, IA (US)

(21) Appl. No.: 19/178,834

(22) Filed: Apr. 14, 2025

Related U.S. Application Data
(60) Provisional application No. 63/769,497, filed on Mar. 10, 2025.

(51) Int. Cl. A01N 37/16; A01N 25/04; A01N 25/22; A01N 25/30; A01N 37/02; A01N 37/04; A01P 1/00

(52) U.S. Cl. CPC A01N 37/16; A01N 25/04; A01N 25/22; A01N 25/30; A01N 37/02; A01N 37/04; A01P 1/00

(57) ABSTRACT

The present invention relates to the use of compositions containing an effective amount of peroxyacid to reduce the viral load in animal feed and water supply, as well as the surfaces and containers in which the feed and water come into contact. Another aspect of the present invention relates to a formaldehyde-free antiviral composition suitable for animal feed, water, and associated storage containers, wherein the composition is capable of controlling the growth of viruses, including but not limited to African Swine Fever, PED, and Avian Influenza. Another aspect of the present invention relates to a kit for treating animal feed to control the growth of viruses comprising a peroxypropionic acid concentrate and a buffered solution.

### CROSS-REFERENCE TO RELATED APPLICATIONS

The present application claims the benefit of priority to U.S. Provisional Patent Application No. 63/633,363, filed Apr. 12, 2024, Provisional Patent Application No. 63/665,601, filed Jun. 28, 2024, and Provisional Patent Application No. 63/769,497, filed Mar. 10, 2025, each entitled “COMPOSITIONS CONTAINING PEROXYACIDS TO REDUCE VIRAL LOAD IN ANIMAL FEED AND RELATED METHODS,” the entire disclosures of which are incorporated herein by reference in their entireties.

### BACKGROUND OF THE INVENTION

Contamination of feed products represents a significant concern in the perpetuation of viral infections. Many devastating viruses can spread through the transport and consumption of contaminated feed products. These viruses put a strain on the production of livestock and affect the economics and volume of the food supply.

For example, African Swine Fever (ASF) is a highly contagious hemorrhagic viral disease of domestic and wild pigs, which is responsible for serious economic and production losses around the globe. ASF resembles classical swine fever (CSF) (hog cholera) so closely that laboratory tests are required to differentiate the viruses. ASF is caused by a unique virus of the Asfarviridae family, and which only infects domestic and wild pigs and a variety of soft-bodied ticks of the genus Ornithodoros. The ticks transmit it through all stages of their life cycle and perpetuate the disease. ASF is currently present in wild and/or domestic pigs in the regions of China, Asia, Europe, and Africa.

Pigs and their close relatives, boars and hogs, are the only natural host of the ASF virus, meaning that the virus, to date, has not been shown to harm humans or other animals. Contamination generally occurs via direct contact with tissue and bodily fluids from infected or carrier pigs. It also spreads through transport and consumption of contaminated food products (Contaminated animal feed (Niederwerder, M. C., Risk and Mitigation of African Swine Fever Virus in Feed, Animals (2021) 11 (3), 792; https://doi.org/10.3390/ani11030792)) and in some cases the viral outbreaks have originated from the failure to comply with biosecurity standards by feeding waste food to domestic pigs.

ASF can affect pigs of any age. Clinical signs and mortality rates can vary according to the virulence of the virus and the type or species of pig. Acute forms of ASF are characterized by high fever, depression, anorexia and loss of appetite, hemorrhages in the skin (redness of skin on ears, abdomen and legs), abortion in pregnant sows, cyanosis, vomiting, diarrhea and death within 6-13 days. The mortality rates of pigs acutely infected with ASF may be as high as 100%. Subacute and chronic forms of ASF produce less intense clinical signs that may be expressed for longer periods of time, but with mortality rates still ranging from 30-70%.

ASF has not yet reached or been reported in pigs in the United States. As there is currently no vaccine or treatment for the prevention and control of ASF, the prevention of ASF in countries free of the disease depends on the implementation of appropriate import policies and biosecurity measures, ensuring that neither infected live pigs, nor pork products, are introduced into areas free of ASF. This includes ensuring proper disposal of waste food from aircraft, ships or vehicles coming from affected countries and policing illegal imports of live pigs and pork products from affected countries. However, during outbreaks of ASF in affected countries, the control of ASF can be very difficult.

Another virus with devastating and wide-reaching impact is Avian Influenza (commonly referred to as “bird flu”), where the latest outbreak in the United States has resulted in the culling of an estimated 100 million birds, including an estimated 20 million egg-laying hens as reported by the United States Department of Agriculture (January 2025). The bird flu outbreak has resulted in the shortage of eggs and driven egg prices to historic prices, where it has been reported that eggs have seen a 40% price increase since the beginning of 2025 alone. In many areas, eggs are quickly sold-out, with many restaurants now imposing a “surcharge” for eggs. Controlling the spread of the virus, including through potential sources of contamination such as feed, has become increasingly urgent.

In chickens, highly pathogenic avian influenza can cause gasping, extreme diarrhea, swelling around the head, neck, and eyes, and rapid death. Highly pathogenic avian influenza has historically had a mortality rate in poultry of 75 to 100%. The threat of avian influenza is not limited to birds; in fact, HPAI is known to infect other animals and occasionally humans. It has been suggested that transmission through feedstock poses a relatively low risk for the spread of avian influenza, but there is still a risk that should be managed through biosecurity measures, including screening incoming feed and on-site feed management practices. Azeem, S. et al. (2022), Evaluation of Feedstuffs as a Potential Carrier of Avian Influenza Virus between Feed Mills and Poultry Farms, Pathogens 11 (7), 755.

Yet another concerning virus that affects swine, in particular, is porcine epidemic diarrhea virus (PED). PEDV has been a long-standing concern in the industry since its first occurrence in the 1970s. Wood E. (1977), An apparently new syndrome of porcine epidemic diarrhea, The Veterinary Record, 100 (12): 243-4. PEDV is an enveloped, positive-sense single-stranded RNA virus, that affects swine. Lin C. M. et al. (2016), Evolution, antigenicity and pathogenicity of global porcine epidemic diarrhea virus strains, Virus research, 226:20-39. The disease is highly pathogenic and causes clinical symptoms such as diarrhea, vomiting, and dehydration, leading to huge economic losses due to its high mortality rate in piglets. Jung K. et al. (2020), Porcine epidemic diarrhea virus (PEDV): An update on etiology, transmission, pathogenesis, and prevention and control, Virus research, 286: 198045; Stevenson G. W. et al. (2013), Emergence of Porcine epidemic diarrhea virus in the United States: clinical signs, lesions, and viral genomic sequences, Journal of veterinary diagnostic investigation, 25 (5): 649-654. Although much effort historically has focused on vaccine production, starting in 2010, a new variant of highly pathogenic GII PEDV appeared in southern China, which rendered the vaccines and drugs available for classic PED ineffective. Due to its resistance to current treatment methods the new variant and subsequent subvariants increased the average morbidity and mortality rate of the disease.

PED is highly contagious. Upon its first case in the United States in April 2013, it reportedly spread to 29 neighboring states within 13 months. Hennessy, D. (2014), Hog Markets and the Porcine Epidemic Diarrhea Virus. Agricultural Policy Review, Spring 2014, Center for Agricultural and Rural Development, Iowa State University; Schulz L. L. and Tonsor G. T. (2015), Assessment of the economic impacts of porcine epidemic diarrhea virus, United States. Journal of Animal Science, 93 (11): 5111-51189. The spread killed at least eight million piglets in the United States. Porcine epidemic diarrhea virus: an emerging and re-emerging epizootic swine virus, Virology journal. 12:1-16. In 2014, piglet mortality in Germany was more than 70%; from 2016-2018, piglet morality was 80-95% in Mexico. Zhang Y. et al. (2022), Porcine Epidemic Diarrhea Virus: An Updated Overview of Virus Epidemiology, Virulence Variation Patterns and Virus-Host Interactions, Viruses, 14 (11): 2434. Thus, PED has become a serious threat to the global swine industry, with a presence in Asia, North America, and Europe. PED virus can be spread through direct or indirect contact, including through contaminated equipment and feed. Dee S. A. et al. (2018), Survival of viral pathogens in animal feed ingredients under transboundary shipping models, PloS one. 13 (3), e0194509. Thus, strict biosecurity and pathogen mitigation measures are needed to minimize the spread of this disease.

There is therefore a need in the agricultural industry for an effective means of identifying, preventing, and treating viruses that are transmitted via feed, water, and contaminated surfaces (including, for example, storage containers), and pose a significant threat to the food supply, for instance ASF, Avian Influenza, and PED. Antimicrobial agents are often added to animal feed and ingredients, but the current options present in the art each have drawbacks. Furthermore, as a person of ordinary skill in the art would recognize, antimicrobials are generally not universally effective in killing all types of microbes (i.e., viruses, bacteria, fungi, molds).

Conventionally, formaldehyde has used in animal feed to control the spread of bacteria, such as Salmonella, and more recently has been identified to mitigate against the spread of viruses, such as ASF and PED viruses. However, globally, there are rising concerns about its toxicity. Its use was banned in the European Union in 2018, and South Korea, Japan, and Taiwan followed suit, banning the use of formaldehyde in feed in their countries. Furthermore, for formaldehyde to be effective against all of its desired targets, it generally must treat feed for 1 to 3 days, which can be an inconvenient waiting period. Due to the rising concerns associated with formaldehyde, there remains a need for suitable alternatives to formaldehyde that are capable of mitigating against the risk of viral contamination in feed. Since countries have started banning the use of formaldehyde, much effort has been invested into an effective formaldehyde replacement to combat microbes in feed, but these solutions so far have not provided an adequate replacement, whether due to performance or cost.

Previous studies have also demonstrated the effectiveness of medium chain fatty acids (MCFA) and derivatives against common bacteria such as Salmonella spp., Escherichia coli, Staphylococcus aureus, Listeria monocytogenes and Campylobacter jejuni (Jackman et al., 2020), (Skrivanová et al., 2004), (Skrivanová et al., 2005), (Marounek et al., 2003), (Batovska et al., 2009). In addition, Cochrane et al. (2020) and Tran et al. (2021) demonstrated the efficacy of MCFA in mitigating porcine epidemic diarrhea (PED) and African swine fever (ASF) viruses, respectively. But, the relative costs of using MCFA are high.

More recently, it has been suggested that formic acid may have a potential antiviral effect via DNA depurination. However, when applied in feed, a higher effective dosage was required to reduce viral infectivity to a comparable level as Sal CURB RME Liquid (a formaldehyde-based product).

Peracetic acid, also known as peroxyacetic acid or PAA, has been used as a disinfectant in waste-water treatment (Baumeister, L. J., 2008) (Kitis, M., 2004) and food industries (Stearns et al., 2022) due to its effectiveness against microbial contamination. But while it has been reported that PAA possesses strong antimicrobial activity, it has been reported that PAA is ineffective at inactivating some viruses and does not inactivate all viruses to the same degree. Fraisse A, Temmam S, Deboosere N, Guillier L, Delobel A, Maris P, Vialette M, Morin T, Perelle S. (2011), Comparison of chlorine and peroxyacetic-based disinfectant to inactivate Feline calicivirus, Murine norovirus and Hepatitis A virus on lettuce, Int J Food Microbiol, 151:98-104. doi: 10.1016/j.ijfoodmicro.2011.08.011; Allwood P B, Malik Y S, Hedberg C W, Goyal S M. (2004), Effect of temperature and sanitizers on the survival of feline calicivirus, Escherichia coli, and F-specific coliphage MS2 on leafy salad vegetables, J Food Prot 67:1451-1456. doi: 10.4315/0362-028x-67.7.1451; Lukasik J, Bradley M L, Scott T M, Dea M, Koo A, Hsu W Y, Bartz J A, Farrah S R. (2003), Reduction of poliovirus 1, bacteriophages, Salmonella montevideo, and Escherichia coli O157:H7 on strawberries by physical and disinfectant.

In view of the limitations discussed above, researchers continue to search for a broad-spectrum antiviral, where the ideal composition would serve as a mitigant against viruses and capable of being added to an animal's diet or water supply or applied to the surfaces that come into contact with an animal's feed or water.

### BRIEF SUMMARY OF THE INVENTION

Although various studies have been conducted with peroxyacids in other contexts, to the inventors' knowledge, the present invention represents the first time that compositions containing various peroxyacid combinations have been studied as a feed additive for controlling or inhibiting the spread of viruses in animal feed, including but not limited to viruses such as ASF, PED, Avian Influenza, and other viruses. This may due to the hesitation in using peroxyacid in feed mills, where corrosion of the feed mill equipment, primarily mild steel material, poses a risk.

Here, the inventors have surprisingly discovered a feed additive that contains one or more peroxyacid, such as perpropionic acid (also referred to herein as peroxypropionic acid or PPA) is capable of controlling or inhibiting the spread of viruses in animal feed, including but not limited to viruses such as ASF, PED, foot-and-mouth disease virus (FMDV), Senecavirus A (SVA), which is a surrogate for FMDV, bovine viral diarrhea virus (BVDV), Avian Influenza virus, influenza A virus of swine (IAV-S), bovine herpesvirus type 1 (BHV-1), porcine reproductive and respiratory syndrome virus (PRRSV), and avian paramyxovirus Newcastle disease virus (NDV).

The present invention relates to the use of compositions containing an effective amount of peroxyacid to reduce the viral load in animal feed and water supply, as well as the surfaces and containers in which the feed and water come into contact. Another aspect of the present invention relates to a formaldehyde-free antimicrobial composition suitable for animal feed, water, and associated surfaces (such as storage containers), wherein the composition is capable of controlling the growth of viruses, including but not limited to African Swine Fever, PED, and Avian Influenza.

### BRIEF DESCRIPTION OF THE FIGURES

FIG. 1 depicts the chemical structure of representative peroxyacids.

FIG. 2 depicts the method-based cell culture used to evaluate the virucidal potential of PPA (in phosphate buffer, neutralizers) in animal feed.

FIG. 3 depicts the virucidal potential of PPA (in phosphate buffer, neutralizers) in animal feed.

FIGS. 4-9 demonstrate the efficacy of PPA compositions at different concentrations using TCID50 and PCR.

FIG. 10 is an overview of the A) healthy PAM cells and B) unhealthy PAM cells due to cytotoxicity through an inverted microscope, without the addition of red blood cells.

FIG. 11 is an overview of the A) healthy PAM cells and B) ASFV-infected PAM cells through an inverted microscope after two days of incubation with red blood cells.

FIG. 12 is an overview of the A) healthy MDCK cells, cell death due to B) treatment solution and C) viral infection (cytopathic effect).

FIG. 13 demonstrates the antiviral activity of PPA and SC LF Liquid against PEDV, where FIG. 13A demonstrates phosphate-buffered saline (PBS) at 5, 10, 20, and 100 ppm, and FIG. 13B demonstrates feed extract at 20, 100, and 500 ppm. The feed extract was prepared by stirring the feed with PBS at a 1:2 ratio and filtering through a 0.2 mm filter to remove particulates. The detection limit for both assays is 1.7 log (TCID₅₀/mL).

FIG. 14 demonstrates the effect of PPA on RNA and viral structure, where FIG. 14A demonstrates the effect of PPA on RNA and FIG. 14B demonstrates the viral structure by determining the quantification cycle using RT-qPCR at 0, 500, 2500, and 12500 ppm.

FIG. 15 demonstrates the antiviral effect of PPA plus phosphate buffer and PROSIDIUM plus phosphate buffer in PEDV-contaminated animal feed using RT-qPCR when treated with the two products at 0, 200, 500, and 1000 grams/MT of PEDV-contaminated feed.

### DETAILED SUMMARY OF THE INVENTION

As described above, ASF is highly contagious with high mortality rates, resulting in high economic losses globally. Chemical feed additives such as formaldehyde-based solutions and medium chain fatty acids (MCFA) are commonly used as mitigants to reduce the risk of ASF. Niederwerder, M. C. (2021). Risk and Mitigation of African Swine Fever Virus in Feed. Animals, 11:792. For instance, it has been shown that Sal CURB RME Liquid (Kemin Industries, Des Moines, Iowa), a formaldehyde-based product, could effectively reduce the viral load of ASF virus in feed. However, there have been increasing concerns over formaldehyde use and the high cost of MCFA.

In contrast to previously known solutions, the present invention relates to the use of compositions containing one or more peroxyacids to control the growth or spread of viruses in animal feed and water, as well as surfaces that come into contact with feed or water, including storage containers. In at least one embodiment, the present invention relates to controlling the growth or spread of viruses by reducing the viral load in animal feed.

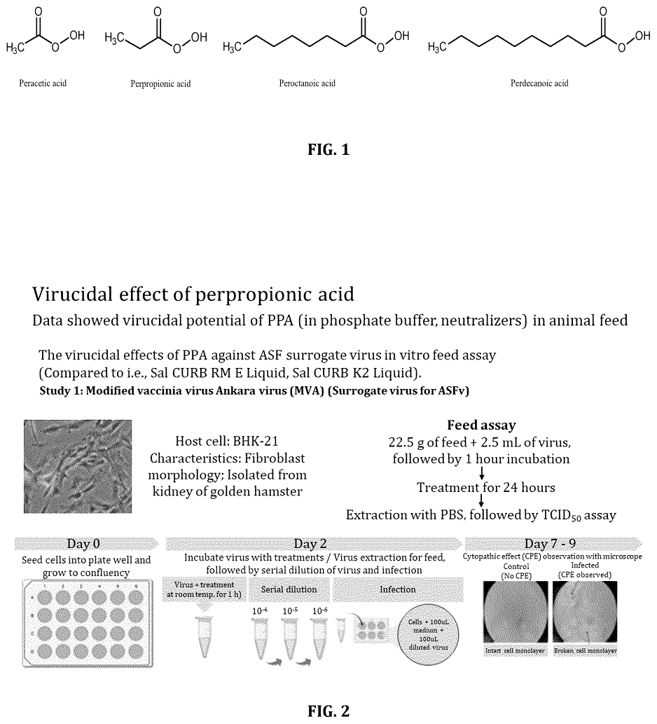

Peroxyacids (FIG. 1) are strong oxidizing agents that have been used as disinfecting agents in meat industries and surface cleaning applications of medical instruments due to their ability to denature proteins and disrupt the cell wall membrane of microorganisms. However, to the inventors' knowledge, peroxyacids have not previously been considered as a mitigant against viruses in feed, for instance as a feed additive with anti-viral properties that could be added to animal feed.

One reason for the hesitancy around using these compounds could be that peroxyacids react violently when in touch with mild steel or carbon steel, which is predominantly the material of construction of mixer units in feed mills. These mixers are used for blending various micro and macro feed components. Therefore, this poses a serious corrosion risk when the peroxyacid product is sprayed onto the feed ingredients while mixer is in operation. Corrosion of mixer components could result in equipment malfunction and damage, which would be detrimental to the feed mill, or contamination of the feed by metal flakes or metal pieces, which would be detrimental to the feed product. The inventors surprisingly discovered that the integration of peroxyacids by the feed mills did not result in any damage to the feed mill or contamination of the feed product.

Another reason could be that peroxyacids are, by their nature as an oxidizing agent, extremely volatile. This volatility makes these compounds difficult to manage. Shipping, handling, and storage of these compounds has to be carefully considered and requires many safety standards to be in place to prevent excess degradation of the compounds and also to prevent any adverse reactions that could pose a danger to people or property.

Perpropionic acid (PPA) solution, for instance where the PPA solution comprises PPA, hydrogen peroxide, propionic acid, and water, was recently reported to be effective against Salmonella in feed applications and could potentially replace the traditional Salmonella mitigants, as described by the inventors in U.S. Application Ser. No. 63/525,315, which is expressly incorporated in its entirety herein. For example, it was reported that a 20% PPA solution demonstrated a comparable Salmonella-killing effect as formaldehyde-based Sal CURB which is commonly regarded as a Salmonella mitigant. Although efficacy of PPA against Salmonella bacteria has been demonstrated, to the inventors' knowledge, this disclosure represents the first-known study of the virucidal effect of PPA, including specific testing for modified vaccinia virus Ankara (MVA) (which is commonly used as the surrogate virus for African swine fever (ASF)), porcine epidemic diarrhea (PED) virus, African swine fever virus (ASFV), and high pathogenic avian influenza virus (AIV).

As used herein, PROSIDUIM™ refers to Kemin Industries' branded product containing PPA. PROSIDUIM™ comprises two components that are mixed on site, prior to application. The two components are PROSIDIUM C and PROSIDIUM S. PROSIDIUM C refers to the PPA concentrate, containing PPA at approximately 15-20%, for instance 16-17%, as well as propionic acid at 40-50%, acetic acid at 5-12% and hydrogen peroxide at 5-10%. PROSIDIUM S refers to the buffer that can be used in combination with the concentrate, containing phosphoric acid and sodium hydroxide. According to at least one embodiment, the components are mixed at a C:S ratio of 9:1.

According to at least one embodiment, the composition is an animal feed additive comprising an effective amount of one or more peroxyacids, for instance a buffered peroxyacid solution, to control or inhibit the spread of viruses in animal feed, including but not limited to viruses such as ASF, PED, FMDV, SVA, BVDV, AIV, IAV-S, BHV-1, and PRRSV.

According to at least one embodiment, the compositions of the present invention contain an effective amount of PPA to control the growth of viruses, such as ASF, PED, and AIV. In certain embodiments, the compositions contain an effective amount of PPA and at least one organic acid, such as acetic acid to control the growth of viruses. In certain embodiments, the composition further comprises propionic acid.

Another aspect of the present invention relates to a formaldehyde-free composition suitable for animal feed that is capable of controlling the growth of viruses, including but not limited to ASF virus, PED virus, and avian influenza virus.

Another aspect of the present invention relates to using the compositions containing peroxyacids to reduce any type or form of African Swine Fever (ASF) disease in swine caused by the African Swine Fever virus (ASFV), a large double stranded DNA virus in the Asfarviridae family, via feed and water as vectors.

According to at least one embodiment, the present invention relates to using the compositions containing one or more peroxyacids in an amount sufficient to reduce any type of virus, for instance in an animal's diet, water supply, or containers or surfaces that come into contact with an animal's diet or water supply.

According to at least one embodiment, the present invention relates to using the compositions containing one or more peroxyacids as an antiviral, for instance killing any viruses that may be present in an animal's diet, water supply, or containers or surfaces that come into contact with an animal's diet or water supply.

According to at least one embodiment, the present invention relates to using the compositions containing one or more peroxyacids in an amount effective to inactivate any virus that may be present in an animal's diet, water supply, or containers or surfaces that come into contact with an animal's diet or water supply.

According to at least one embodiment, the composition contains a combination with other organic peroxides (i.e., perester, peracetic acid, peroctanoic acid) in amounts effective to reduce the viral load in vectors, such as animal feed or water supply. According to at least one embodiment, the composition contains an amount effective to reduce the viral load to an undetectable amount.

According to at least one embodiment, the composition of the present invention is an animal feed additive comprising an effective amount of peroxyacid, for instance a buffered peroxyacid, to reduce the viral load present in animal feed.

Another aspect of the present invention relates to a formaldehyde-free composition suitable for animal feed that is capable of controlling the growth of viruses, including but not limited to ASF virus.

Another aspect of the present invention relates to a formaldehyde-free composition suitable for animal feed that is capable of controlling the growth of viruses, including but not limited to PED virus.

Another aspect of the present invention relates to a formaldehyde-free composition suitable for animal feed that is capable of controlling the growth of viruses, including but not limited to avian influenza virus.

According to at least one embodiment, the composition of the present invention surprisingly demonstrated efficacy in mitigating against the spread of viruses that pose a risk to livestock.

According to at least one embodiment, the present invention relates to methods of reducing an outbreak or spread of a viral disease in livestock comprising adding to animal feed or water supply a composition comprising one or more peroxyacids, wherein the composition reduces the viral load of the animal feed, where the viral outbreak includes but is not limited to ASFV, PEDV, FMDV, SVA, BVDV, Avian Influenza virus, IAV-S, BHV-1, and PRRSV.

In alternative embodiments, the present invention relates to a feed additive for controlling or inhibiting the spread of viruses present in animal feed, including but not limited to viruses such as ASFV, PEDV, FMDV, SVA, BVDV, Avian Influenza virus, IAV-S, BHV-1, and PRRSV.

According to at least one embodiment, the composition does not contain formaldehyde.

According to at least one embodiment, the composition does not contain formic acid.

According to at least one embodiment, the composition does not contain butyric acid.

According to at least one embodiment, the composition does not contain propionic acid.

According to at least one embodiment, the composition does not contain acetic acid.

According to at least one embodiment, the composition of the present invention optionally contains at least one surfactant.

According to at least one embodiment, the composition of the present invention optionally contains at least one emulsifier. Suitable emulsifiers for this purpose include, but are not limited to, glycerol monooleate, soya lecithin, glycerin monostearate, potassium stearate, calcium stearoyl lactylate (CSL), DATEM, glyceryl monostearate, mono propylene glycol, SPAN 80, sodium stearoyl lactylate (SSL), Tween, sodium stearate, glycerol triacetate, sugar esters, non-dairy creamer, calcium stearate, polyglycerol polyricinoleate (PGPR), lecithin, mono and diglycerides, monoglyceride derivatives, polyglycerol esters (PGE), propylene glycol esters (PGMS), sucrose esters, and sorbitan esters and polysorbates.

According to at least one embodiment, the compositions of the present invention are suitable for adding to animal feed and can be combined with known animal feed ingredients, including but not limited to corn meal, soybean meal, fish meal, soy oil cake, dried distillers' grains with solubles (DDGS), etc.

According to at least one embodiment, the composition is used to disinfect containers for feed, for example grain bins (storage of dry corn and soybeans), silos (storage of silage, grass or harvested green and wet to feed dairy cattle), storage systems or containers that provide feed or water supply to animals.

According to at least one embodiment, the composition is a dry ingredient. For instance, in at least one embodiment, the composition is a feed additive that is incorporated into the animal feed at the feed mill.

According to at least one embodiment, the composition is a wet ingredient. For instance, in at least one embodiment, the composition is a feed additive that is capable of being sprayed onto the feed. In at least one embodiment, the composition is applied to the animal feed using a liquid applicator.

According to at least one embodiment, the composition is composed of a first component and a second component. In at least one embodiment, the first component comprises at least one peroxyacid, for instance a concentrate, and the second component comprises a buffer.

When combined, the first component and the second component create a buffered solution with a pH in the range of <1 to 0, for example pH 1-4, or more specifically 2-3. In at least one embodiment, the first component has a pH of less than 2, for instance less than about 1, and the second component has a pH in the range of about 12 to 14, for instance about 12 to 13. In at least one embodiment, the components are mixed on site to form the composition prior to application.

According to at least one embodiment, the composition is composed of a first component and a second component. In at least one embodiment, the first component comprises at least one peroxyacid and the second component comprises a buffer. In at least one embodiment, the first component has a pH less than 1 and the second component has a pH in the range of 12-13. In at least one embodiment, the components are mixed on site to form the composition prior to application.

According to at least one embodiment, the first component and the second component are combined in a ratio ranging from 4:1 to about 12:1, for instance about 7:1, 8:1, 9:1, 10:1, 11:1 or 12:1. In at least one embodiment, the first component and the second component are combined in a ratio of about 9:1.

According to at least one embodiment, the present invention relates to methods of treating animal feed with a mitigant for viruses, wherein the feed can be treated within a shorter treatment time compared to conventional methods for mitigating against the spread of viruses. For instance, in at least one embodiment, the treatment time is one hour or less. In certain embodiments, the treatment time is about one hour or less, about 30 minutes or less, about 20 minutes or less, about 10 minutes or less, or about 5 minutes or less. In certain embodiments, compositions of the present invention are capable of killing viruses in one hour or less.

According to certain embodiments, the composition contains one or more peroxyacids. In alternative embodiments, the composition contains two or more peroxyacids, including PPA. In alternative embodiments, the composition contains PPA (plus buffer), with efficacy that is superior to other organic peroxides, such as treatments with peracetic or percarbonate alone.

In at least one embodiment, the composition of the present invention provides desirable characteristics to the animal's diet when used as a food or feed additive, such as providing a prolonged or extended shelf-life of the food or feed, where the inhibition of viruses assists in providing a longer shelf-life for the treated product, such as food or feed, compared to an untreated product (i.e., a product that has not been treated with compositions of the present invention).

For purposes of this disclosure, “animal feed” may be provided to the animals or livestock through any convention means well known to persons skilled in the art, include but not limited to top-dress, mixed in by hand, pelleted, mixed with crumbles or granular solids, etc.

### EXAMPLES

The inventors provide the following examples, which aim to evaluate the virucidal effect of PPA and compare the virucidal efficacy of PPA against other compositions and commercially-available products.

### Example 1: Efficacy of PPA Compositions on MVA Inactivation

### Background

Testing for efficacy against ASF proves to be challenging, as such studies often require a long testing period due to reliance on external institutes and high testing costs, where studies with ASF can only be conducted in high-level containment laboratories (Biosafety level three (BSL-3)). In view of these restrictions, MVA, an enveloped virus with a double-stranded DNA genome, has been previously validated as a suitable BSL-2 surrogate virus for ASF virus. Rhee C. H., Her M., Jeong W. (2022), Modified Vaccinia Virus Ankara as a Potential Biosafety Level 2 Surrogate for African Swine Fever Virus in Disinfectant Efficacy Tests, Pathogens, 11:320. For instance, Rhee et al. compared the susceptibility of MVA and ASF viruses to disinfectants and validated the suitability of MVA as a potential BSL-2 surrogate virus. Similarly, Tanneberger et al. reported the similarities between MVA and ASF viruses in efficacy screening. Although there have been various efficacy screenings of mitigants and disinfectants conducted against ASF and/or MVA, to the inventors' best knowledge, no study has been conducted with PPA against these viruses for feed application. Tanneberger F., Abd El Wahed A., Fischer M., Blome S., Truyen U. (2021), The efficacy of disinfection on modified vaccinia Ankara and African swine fever virus in various forest soil types, Viruses, 31:2173.

### Materials and Methods

Cell culture and virus. MVA, purchased from American Type Culture Collection, (ATCC; VR-1566) was propagated with BHK-21 fibroblasts (ATCC; CCL-10) to obtain a viral titer of ca. 10⁷ TCID₅₀/mL. The propagation was carried out at a multiplicity of infection (MOI) of 0.1 at 37° C. and 5% CO₂ in Eagle's minimum essential medium (EMEM, ATCC; 30-2003) supplemented with 10% fetal bovine serum (FBS; Hyclone Laboratories Inc, 30071.03). Virus titration was subsequently carried out to determine the viral titer.

Virucidal effect of treatments in solution. The virucidal effect of PPA was preliminarily evaluated with other existing Kemin products such as Sal CURB RME Liquid (M804403) and Sal CURB K2 Liquid (M804409) in solution. Compositions of each treatment group were tabulated in Table 1. A volume of 99 μL virus solution (ca. 10⁷ TCID₅₀/mL) was treated with 1 μL of the test solutions (prepared at 500 g/T in phosphate-buffered saline (PBS; Hyclone Laboratories Inc, SH30256.01) at room temperature for one hour. After one hour, the treatments were neutralized with 25 μL 0.1 M sodium thiosulphate (Merck KGaA, 1.09147.1000) and serial diluted up to 10⁻⁷ with EMEM supplemented with 10% FBS prior to virus titration.

TABLE 1
Compositions of treatment groups

| Sample | Composition/RM code |
|---|---|
| PPA | 21.8% PPA and 8.3% hydrogen peroxide |
| Sal CURB K2 | 50% formic, 9% propionic and 7% lactic |
| Liquid | acids/M804409 |
| Sal CURB RM E | 33% formaldehyde and 13% propionic |
| Liquid | acid/M804403 |

Virucidal effect of treatments in feed. Soybean and corn were procured from a local supplier, N & N Agriculture Pte Ltd. The raw materials were ground and mixed to prepare a feed composite in a ratio of 7:3 corn and soybean. The feed composite was autoclaved at 121° C. for one hour and dried at 65° C. over 24 hours prior to use. The contaminated feed was prepared by adding 1 mL of virus solution (containing ca. 10⁷ TCID₅₀/mL) into five grams of sterile feed composite, pre-weighed in a 50 mL centrifuge tube. The contaminated feed was homogenously mixed with a sterile loop to break up any clumps and left to stand for 10 minutes. Subsequently, individual portions of the contaminated feed were separately treated with 500 μL of PPA, Sal CURB K2 Liquid, and Sal CURB RME Liquid at 2.5 kg/T for one hour to evaluate their relative virucidal effect in feed. Briefly, 12.5 mg of the treatment was weighed into a 1.5 mL microcentrifuge tube and topped up to 500 μL with sterile water, prior to adding into the five grams of contaminated feed (pre-weighted in a 50 mL centrifuge tube) with a micropipette. The treated feed samples were homogenously mixed with a sterile loop to break up any clumps. An additional treatment group was prepared with PPA at 1 kg/T to evaluate its effectiveness at low dosage. After one hour of treatment, the treatments were neutralized with 2 mL of 0.1 M sodium thiosulphate and 8 mL of phosphate buffer, achieving a final volume of 10 mL. The feed suspension was vortexed to mix homogenously for 20 seconds and subsequently centrifuged at 1200 g for 10 minutes. The supernatant was filtered through a 0.45 um syringe filter (Sartorius Stedim Biotech Gmbh, 16555) and serial diluted up to 10⁻⁶ with EMEM supplemented with 10% FBS prior to virus titration. Blank feed was prepared with 1 mL of EMEM supplemented with 10% FBS and 500 μL of sterile water in replacement of virus and treatment solutions respectively. Negative control was prepared by the addition of 500 μL of sterile water (in replacement of treatment solution) to five grams of contaminated feed. Blank and negative control were subsequently prepared in the same way as the treatment groups.

Virus titration. The loss of infectivity/viability of MVA was determined via the 50% tissue culture infectious dose (TCID₅₀). A 100 μL volume of the ten-fold serial dilutions prepared was inoculated to monolayers of BHK-21 cells seeded in a 96-well plate (Corning Incorporated, 3599) using four wells per dilution. The monolayer of BHK-21 cells was prepared by seeding 100 μL of BHK-21 cells at a concentration of 0.20-0.25 million cells per mL in each well and allowed to grow for 48 hours. The inoculated plates were subsequently incubated at 37° C. in a 5% CO₂ atmosphere for five days. Cytopathic effect (CPE) was observed after five days of incubation. Following this, TCID₅₀ was calculated using the Reed-Muench method, as described in the following references, which are incorporated in their entirety herein: Reed L. J., Muench H. (1938), A simple method of estimating fifty percent endpoints, American Journal of Epidemiology, 27 (3): 493-497; Lei C., Yang J., Hu J., Sun X. (2021), On the calculation of TCID50 for quantification of virus infectivity, Virologica Sinica, 36:141-144.

### Results

Virucidal effect of PPA in solution. The average viability of MVA after one-hour treatment was tabulated in Table 2. PPA is efficacious in reducing the viability of MVA, wherein PPA could reduce MVA to an undetectable level in solution (<2.0 log (TCID₅₀/mL)) at 0.5 kg/T. On the other hand, no statistical differences were observed between Sal CURB RME Liquid, Sal CURB K2 Liquid, and the negative control. This indicates that both Sal CURB RME and K2 Liquid demonstrated negligible virucidal effects.

TABLE 2
Average viability (n = 3) of MVA after treatment in solution.

| | | Virus titer recovered, |
|---|---|---|
| | Dosage | Average ± SD, log |
| Treatment | (kg/T) | (TCID₅₀/mL) |
| Negative control | 0.5 | 4.83 ± 0.29^{a} |
| PPA | 0.5 | <2.00 |
| Sal CURB RM E | 0.5 | 4.56 ± 0.10^{a} |
| Liquid | | |
| Sal CURB K2 | 0.5 | 4.67 ± 0.29^{a} |
| Liquid | | |

SD = Standard deviation; Different alphabet indicates statistically significant difference (p < 0.05).

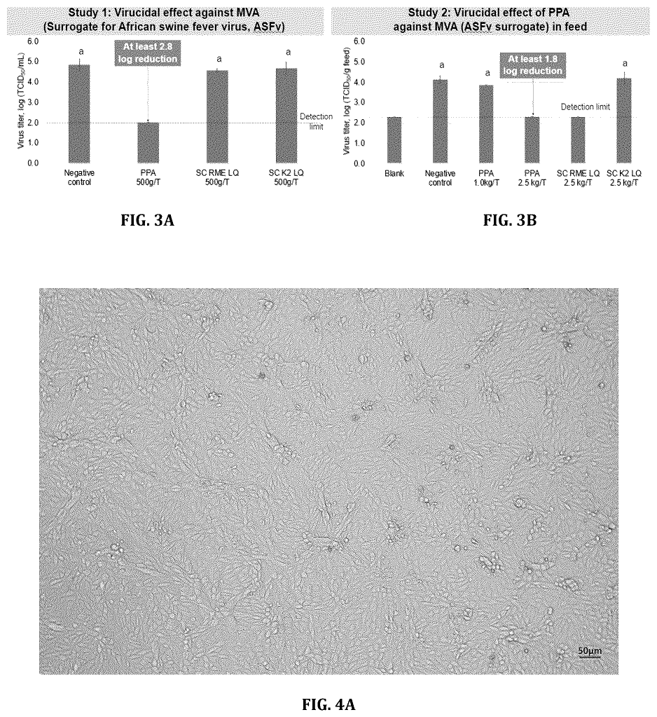

Virucidal effect of PPA in feed. A strong virucidal effect was observed with PPA at 2.5 kg/T where PPA could reduce the virus titer to an undetectable level in feed (<2.30 log (TCID₅₀/mL)) (Table 3, FIGS. 2-3). Comparable virucidal effects were observed with PPA and Sal CURB RME Liquid where negligible infectivity could be detected at 2.5 kg/T, indicating a 1.84 log (TCID₅₀/mL) reduction in virus infectivity. On the other hand, no statistical difference was observed between 1 kg/T of PPA and the negative control, indicating minimal virucidal effect when PPA is applied at 1 kg/T. Consistent with the in-vitro results in solution, Sal CURB K2 Liquid demonstrated negligible virucidal effect against MVA.

TABLE 3
Average viability (n = 3) of MVA after treatment in feed.

| | | Virus titer recovered, |
|---|---|---|
| | Dosage | Average ± SD, log |
| Treatment | (kg/T) | (TCID₅₀/g) |
| Blank | — | <2.30 |
| Negative control | — | 4.14 ± 0.19^{a} |
| PPA | 1.0 | 3.50 ± 0.00^{a} |
| PPA | 2.5 | <2.30 |
| Sal CURB RM E Liquid | 2.5 | <2.30 |
| Sal CURB K2 Liquid | 2.5 | 4.20 ± 0.29^{a} |

SD = Standard deviation; Different alphabet indicates statistically significant difference (p < 0.05).

### Discussion

The virucidal effect of PPA was evaluated against MVA, a validated BSL-2 surrogate virus of ASF virus in comparison to two key Sal CURB products, Sal CURB RME Liquid (formaldehyde-based) and Sal CURB K2 Liquid (organic acid-based) (Table 1).

The preliminary in-vitro efficacy screening was conducted in solution and revealed the strong virucidal effect of PPA where PPA at 0.5 kg/T could reduce MVA infectivity to an undetectable level (Table 2). Surprisingly, Sal CURB RME Liquid showed a negligible virucidal effect on MVA. Such observation could be due to the short treatment period, which was limited to one hour. A neutralizer (to preserve feed and act as an anticorrosive) such as phosphate buffer was used to neutralize the effect of the treatments after one hour. On the other hand, the previous reports on the effectiveness of Sal CURB RME Liquid mainly focused on a longer treatment time (i.e., 1-14 post-treatment days). Following these positive results, the virucidal effect of PPA was further evaluated in feed raw material composite (containing 7:3 corn and soybean matrices). Although PPA demonstrated a strong virucidal effect at 0.5 kg/T in solution, a lower degree was observed at 1 kg/T in feed (p>0.05) (Table 3). This could be due to the high reactivity of PPA, where PPA could react with the feed matrices, increasing the effective dosage required. However, at 2.5 kg/T, PPA demonstrated comparable virucidal efficacy as Sal CURB RME Liquid at the same dosage for a longer treatment period. In comparison, a negligible virucidal effect (p>0.05) was observed with Sal CURB K2 Liquid at 2.5 kg/T in feed.

The inventors have unexpectedly discovered that PPA demonstrates a strong virucidal effect (in buffers, neutralizers) without the drawbacks of other solutions. Although a higher effective dose is required when applied in feed, PPA showed a virucidal impact comparable to the positive control, Sal CURB RME-Liquid at 2.5 kg/T when treated longer.

### Example 2: Efficacy of PPA Compositions on Porcine Epidemic Diarrhea Inactivation

### Materials and Methods

A volume of 100 μL PED virus (ca. 105 TCID50/mL) was treated with 900 μL of treatment solution, prepared in 10 mM phosphate-buffered saline for 1 hour at room temperature. All treatments were tested at 500 ppm. After 1 hour of treatment, the treatments were neutralized with 667 μL of 0.1 M sodium thiosulphate. The mixture was mixed well and left to stand for 10 minutes. Subsequently, the mixture was serial diluted up to 10⁻⁵ with Eagle's minimum essential medium (EMEM) supplemented with 0.3% tryptose phosphate broth and 10 μg/mL of trypsin for virus titration assay.

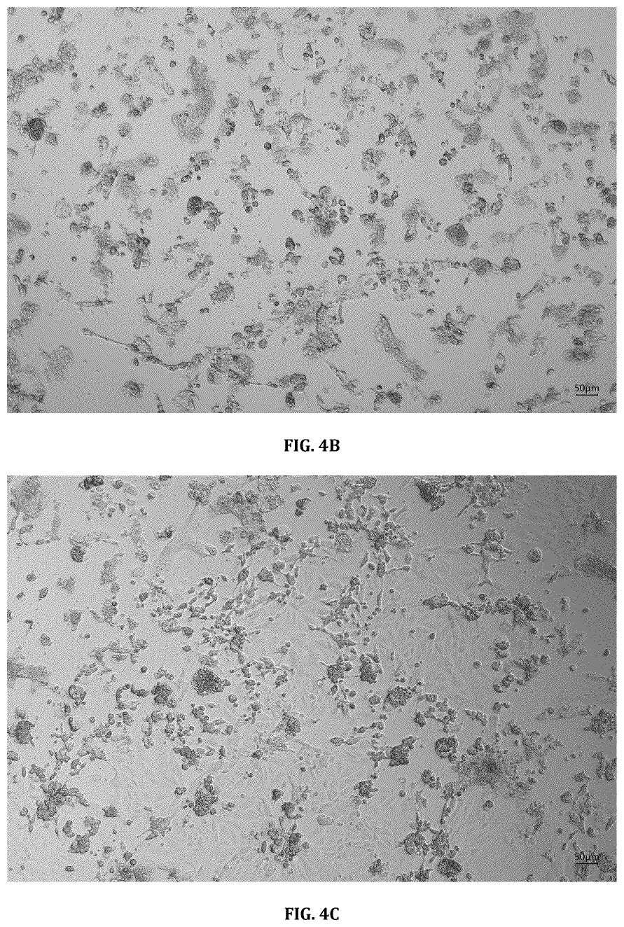

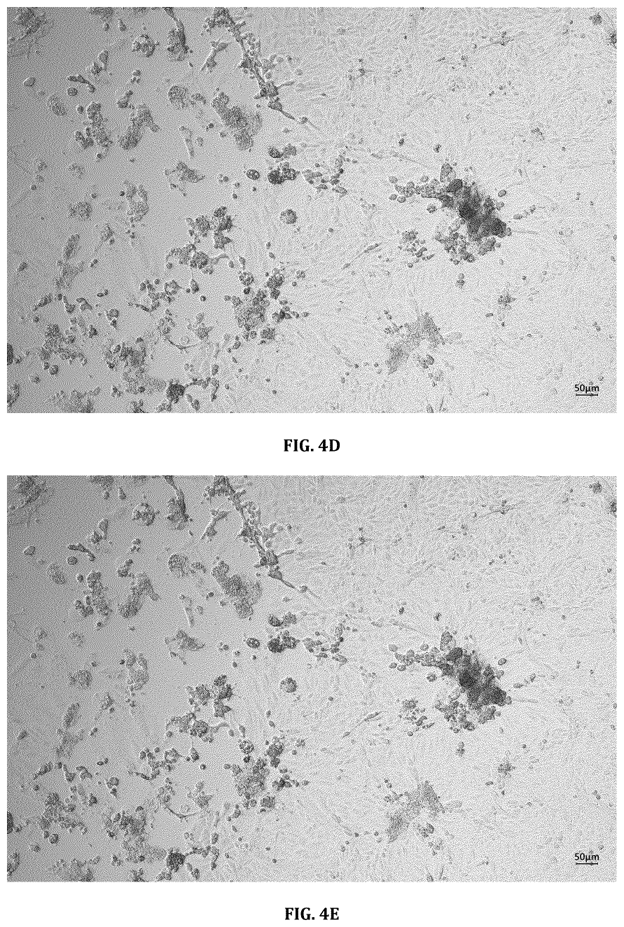

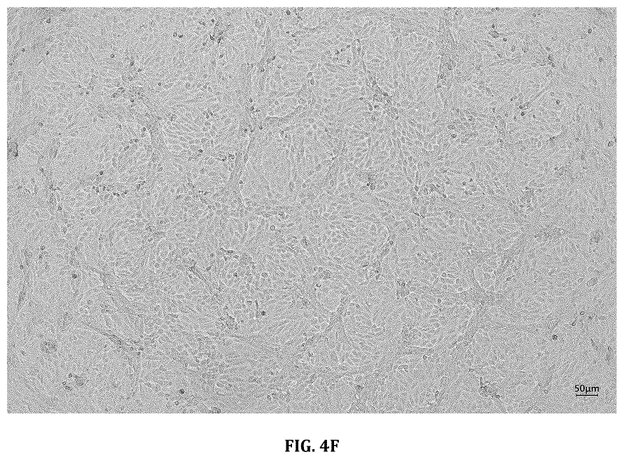

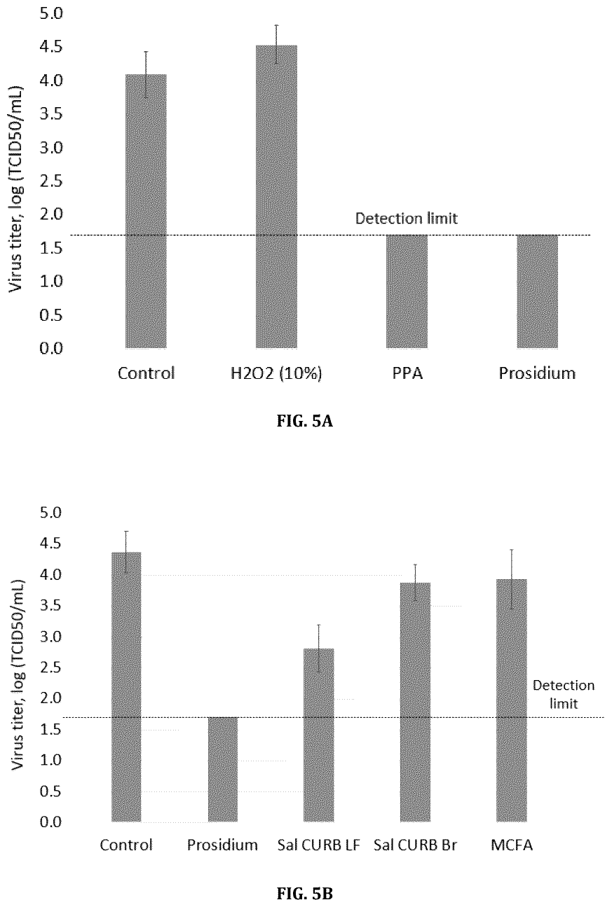

The loss of infectivity/viability of PEDV was determined via the 50% tissue culture infectious dose (TCID₅₀). Briefly, 100 μL of Vero cells were seeded at a density of 2-2.5×10⁵ cells/mL in a 96-well plate and incubated at 37° C. in a 5% CO₂ incubator for 24 hours. The cells are cultured in EMEM supplemented with 10% fetal bovine serum (FBS). After 24 hours, the culture medium in the wells was aspirated and rinsed with phosphate-buffered saline twice. Subsequently, the cells were infected with 200 μL of virus solution. The virus solution was serial diluted by ten-fold by five times before inoculation. Cytopathic effect (CPE) was observed after three days of incubation (FIG. 4). No obvious CPE (clumped cells and broken monolayer) observation. Next, TCID₅₀/mL was calculated using the Reed-Muench method where the detection limit was 1.7 log (TCID₅₀/mL), as summarized in FIGS. 5A-5B. Peroxyacid was shown to be effective against PEDV.

### Results

As shown in FIG. 6, the PPA solution (20 ppm) demonstrated a comparable virucidal effect of Sal CURB LF Liquid (500 ppm) in the culture medium solution.

### Example 3: Evaluating the Inactivation Mechanism of PPA

As shown by Fuzawa et al., peracetic acid (a peroxyacid) may possess strong antimicrobial activity, but it is not effective at inactivating some viruses and does not inactivate all viruses to the same degree. Fuzawa et al. (2020), The Basis of Peracetic Acid Inactivation Mechanisms for Rotavirus and Tulane Virus under Conditions Relevant for Vegetable Sanitation, Appl. Environ. Microbiol. 86 (19). Therefore, it is vital to understand how PPA mechanistically inactivates different viruses under conditions that are relevant to animal feed, in order to design an optimal PPA sanitation practice for virus-contaminated feed.

### Materials and Methods

Viral RNA was extracted from each sample by using genesig Easy RNA extraction kit. The extraction was carried out as per the supplier's recommendation. RNA was eluted from the magnetic beads by using 100 μL of elution buffer. The extracted RNA is subsequently analyzed through qPCR. The amount of extracted RNA was also quantitated with a Qubit™ RNA high-sensitivity assay kit using a Qubit™ 4 Fluorometer. The process and components are summarized in Tables 4 and 5 below.

TABLE 4
RNA Degradation qPCR Protocol

| Step | Cycle Number | Temperature (° C.) | Duration (s) |
|---|---|---|---|
| Reverse | 1 | 55 | 600 |
| transcription | | | |
| Enzyme | 1 | 95 | 60 |
| activation | | | |
| Denaturation & | 45 | 95 | 10 |
| data collection | | 60 | 30 |
| Cooling | 1 | 40 | 30 |

TABLE 5
Components Used in Reaction

| | μL per |
|---|---|
| Component | reaction |
| Luna Universal One- | 10 |
| Step Reaction Mix | |
| (2X) | |
| Nuclease free water | 2.3 |
| Luna WarmStart ® RT Enzyme Mix | 1.0 |
| (20X) | |
| PEDV forward primer (10 μM)^{a} | 0.8 |
| PEDV reverse primer (10 μM)^{b} | 0.8 |
| Extracted DNA sample/ | 5.0 |
| Nuclease free | |
| water as blank | |

^{a}forward primer: 5′-AGT GCT TCA GAG GCT GAT TAC-3′
^{b}reverse primer: 5′-CAC AGT GGG TGG TGT GTA TAA-3′

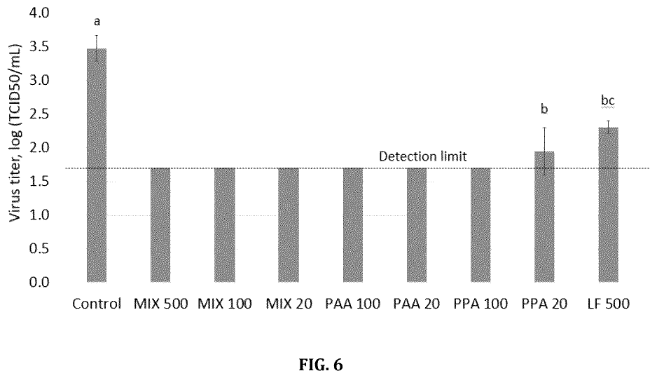

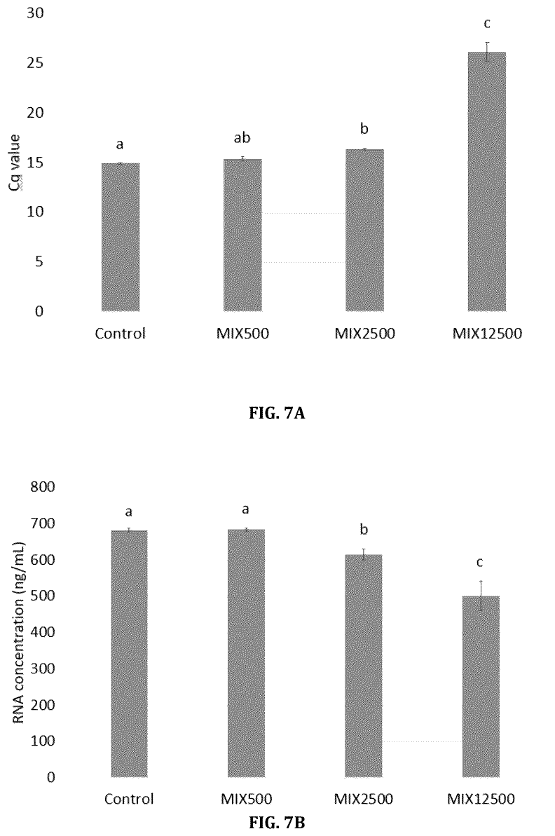

Using this protocol, lower Cq values indicate that a higher amount of RNA is present in the sample. It was observed that at 500 ppm, PPA did not exhibit any RNA damaging effect, while PPA could damage RNA when dosed at 2500 and 12500 ppm, as summarized in FIG. 7A-7B.

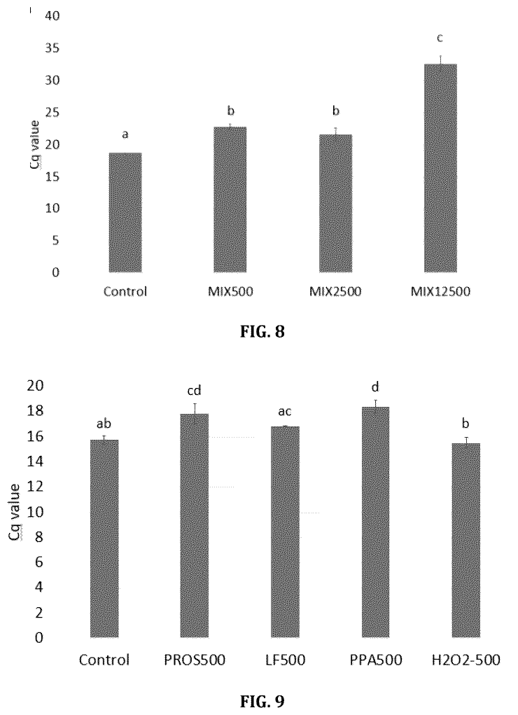

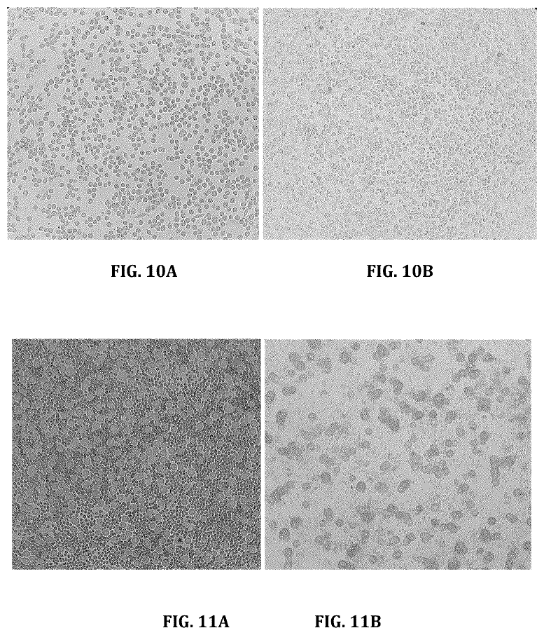

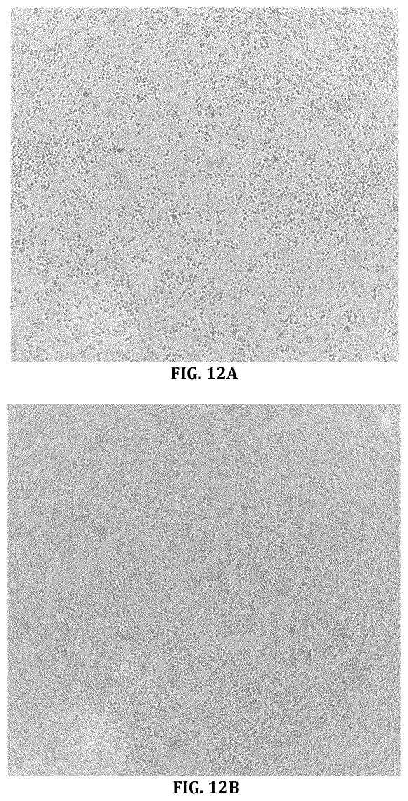

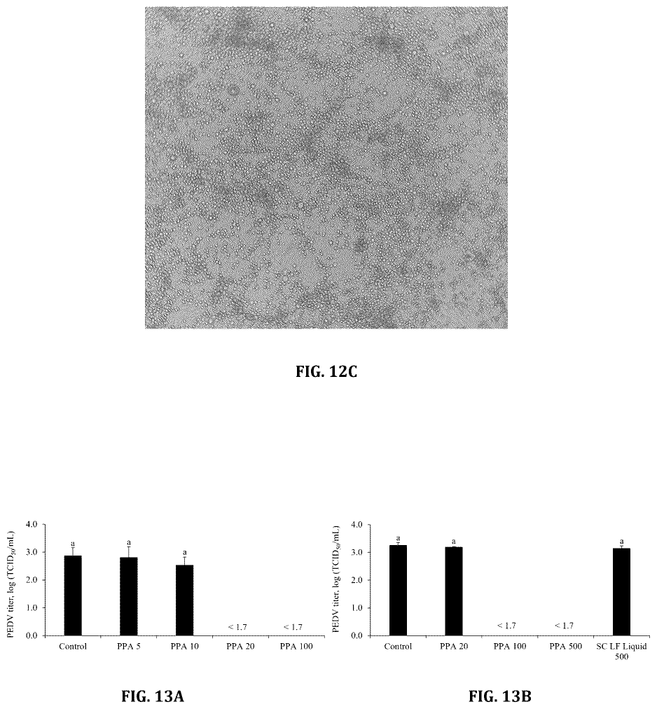

1 μL of RNase A was added to 200 μL of the treated viral samples. The mixture was left to stand at room temperature for 30 minutes. Subsequently, 5 μL of RNase inhibitor was added and left to stand for 10 minutes. 20 μL of proteinase K was subsequently added to the mixture. The mixture was incubated at 37° C. for 30 minutes. After 30 minutes of incubation, RNA was extracted using genesig Easy RNA extraction kit and analyzed through qPCR. A higher Cq value was observed after treatment in comparison to the control, indicating a lower amount of RNA present. The results are summarized in FIG. 8, demonstrating that PPA damages the viral envelope, allowing RNase to access the viral RNA. The effect of the treatment at 500 and 2500 ppm are comparable, and where the effect of treatment at 12500 ppm has the greatest impact on the viral envelope therefore causing the Cq value to be the highest.

As summarized in FIG. 9, the highest Cq value was observed after treatment with PROSIDIUM™ and PPA (500 ppm) compared to the control, indicating a lower amount of RNA present. The results demonstrate that peroxyacid is damaging the viral envelope, allowing RNase to access and degrade the RNA. On the other hand, no significant differences were observed between the Cq values of the control and Sal CURB LF Liquid at 500 ppm, and between control and H₂O₂ at 500 ppm.

Overall, the results showed that peroxyacid treatment could result in viral envelope/capsid damage in the range of 500-12500 ppm. Thus, the virucidal effect observed at 500 ppm via TCID₅₀ assay may be due to the viral envelope/capsid damage.

### Example 4: Efficacy of PPA Compositions on African Swine Fever Virus Inactivation

### Materials and Methods

Cell culture and virus. Pulmonary alveolar macrophage (PAM) and African swine fever virus (ASFV) were used in this study. PAM was cultured in Roswell Park Memorial Institute Medium (RPMI) supplemented with 5% fetal bovine serum (FBS) and 1% antibiotics at 37° C. with 5% carbon dioxide (CO₂). The viral titer of ASFV was at ca. 6 log (HAD₅₀/mL).

Treatments. The study was carried out in solution at a final contamination level of ca. 5 log (HAD₅₀/mL) with treatments and time points as tabulated in Table 6. PROSIDIUM C+S was applied with PROSIDIUM Concentrate and PROSIDIUM S (phosphate buffer solution to temporality stabilize PPA to slow down the aggressiveness of the PPA when in contact with feed mill equipment/mild-steel material) at a 9:1 ratio of the stated concentrations.

TABLE 6
Treatments and assay conditions

| | | Concentration | ASFV | Duration | |
|---|---|---|---|---|---|
| Code | Treatment | (ppm) | HAD₅₀/mL | (h) | Remarks |
| S1 | Sal CURB | <5000 | — | — | Cytotoxicity test |
| | RME Liquid | | | | of disinfectant |
| | SCRME | | | | (2-fold serial |
| S2 | PROSIDIUM | <5000 | — | — | dilution) |
| | C + S | | | | |
| S3 | PPA | <5000 | — | — | |
| S4 | Water | — | — | 0, 0.5, 1, 3, | Negative control |
| | | | | 6 | |
| S5 | Water | — | 10^{6.0} | 0, 0.5, 1, 3, | Positive control |
| | | | | 6 | |
| S6 | Sal CURB | 1000 | 10^{6.0} | 0, 0.5, 1, 3, | — |
| | RME Liquid | | | 6 | |
| | SCRME | | | | |
| S7 | PPA | 1000 | 10^{6.0} | 0, 0.5, 1, 3, | — |
| | | | | 6 | |
| S8 | PROSIDIUM | 200 | 10^{6.0} | 0, 0.5, 1, 3, | — |
| | C + S | | | 6 | |
| S9 | PROSIDIUM | 500 | 10^{6.0} | 0, 0.5, 1, 3, | — |
| | C + S | | | 6 | |
| S10 | PROSIDIUM | 1000 | 10^{6.0} | 0, 0.5, 1, 3, | — |
| | C + S | | | 6 | |
| S11 | PROSIDIUM | 2500 | 10^{6.0} | 0, 0.5, 1, 3, | — |
| | C + S | | | 6 | |
| S12 | PROSIDIUM | 5000 | 10^{6.0} | 0, 0.5, 1, 3, | — |
| | C + S | | | 6 | |

50% hemadsorption dose (HAD₅₀) assay. Treatments were prepared at 1.11 times higher of the stated concentration to account for the dilution upon the addition of virus. The prepared treatment solution was added in 9 parts to 1 part of virus solution. Briefly, 900 μL of the prepared treatment solution (at 1.11 times higher) is added to 100 μL of virus solution. The mixture was vortexed to mix well. Subsequently, the treated virus solution was left to stand till the timepoint stated in Table 6. At each time interval, 250 μL of 0.1 M sodium thiosulphate is added to 1000 μL of the treated virus solution, mix well and leave to stand for 10 minutes for neutralization. After 10 minutes, the solution was serial diluted ten-fold with RPMI only for inoculation into 96-well plates with PAM cells already seeded the day before. 100 μL of the solution at each dilution was inoculated into four wells and incubated for one hour at 37° C. with 5% CO₂. After incubation for one hour, the inoculated solution in each well was removed and replaced with 200 μL of RPMI supplemented with 5% FBS and 1% antibiotics. After 48 hours of incubation, 10 μL of red blood cells were inoculated into each well and further incubated for another 48 hours. HAD₅₀ was calculated using Spearman-Karber method.

Cytotoxicity assay. Treatment was carried out the same way as HAD₅₀ assay. However, instead of virus, 900 μL of treatment solution was added to 100 μL sterile media (to which the virus is stored in) instead of to the virus solution. Subsequently, the mixture was serial diluted two-fold and added to 96-well plate already seeded with PAM cells. Cytotoxic concentration was determined by visually observing the cells using an inverted microscope after 48 hours of incubation at 37° C. with 5% CO₂.

Cytotoxicity. As summarized in Table 7, the maximum non-toxic concentration of Sal CURB RME Liquid (SC RME; a formaldehyde-based product) is the lowest at 156.25 ppm, the maximum non-toxic concentrations of PROSIDIUM C+S and PPA are 1250.00 and 625.00 ppm, respectively. This indicated that Sal CURB RME Liquid is more toxic than PROSIDIUM C+S and PPA to PAM cells.

TABLE 7
The maximum non-toxic concentration of treatments in ppm.

| | | Maximum non-toxic |
|---|---|---|
| | | concentration, |
| Code | Sample | ppm |
| S1 | SC RME | 156.25 |
| S2 | PROSIDIUM C + | 1250.00 |
| | S | |
| S3 | PPA | 625.00 |

SC RME = Sal CURB RM E Liquid (formaldehyde-based product); PROSIDIUM C + S = PROSIDIUM C (concentrate containing PPA 16%) and S (phosphate buffer) applied at 9:1 ratio; PPA = 17% PPA.

50% hemadsorption dose (HAD₅₀) assay. At timepoint 0, the neutralizing solution (i.e., sodium thiosulphate) was added to the treated virus solution within 10-15 minutes. The results showed that PPA and PROSIDIUM C+S can reduce ASFV within a short period of 10-15 minutes of treatment at a concentration as low as 1000 and 200 ppm respectively. On the other hand, the effect of formaldehyde-based product (Sal CURB RME Liquid) is slower; it can only reduce up to 1.25 log (HAD₅₀/mL) of ASFV after 10-15 minutes of treatment. These results are summarized in Table 8.

TABLE 8
In vitro virucidal efficacy of various treatments/products
against African swine fever virus

| | | | ASFV titer after treatment | | | | |
|---|---|---|---|---|---|---|---|
| | | | (log HAD₅₀/mL) | | | | |
| | | | Duration (h) | | | | |
| Code | Sample | Virus | 0 | 0.5 | 1 | 3 | 6 |
| S4 | Water | − | NA | NA | NA | NA | NA |
| S5 | Water | + | 5.5 | 5.5 | 5.5 | 5.5 | 5.5 |
| S6 | SC RME | + | 4.25 | 3.75 | 3.75 | 2.75 | <2.5 |
| | (1000 ppm) | | | | | | |
| S7 | PPA | + | <2.5 | <2.5 | <2.5 | <2.5 | <2.5 |
| | (1000 ppm) | | | | | | |
| S8 | PROSIDIUM | + | <1.5 | <1.5 | <1.5 | <1.5 | <1.5 |
| | C + S | | | | | | |
| | (200 ppm) | | | | | | |
| S9 | PROSIDIUM | + | <1.5 | <1.5 | <1.5 | <1.5 | <1.5 |
| | C + S | | | | | | |
| | (500 ppm) | | | | | | |
| S10 | PROSIDIUM | + | <2.5 | <2.5 | <2.5 | <2.5 | <2.5 |
| | C + S | | | | | | |
| | (1000 ppm) | | | | | | |
| S11 | PROSIDIUM | + | <2.5 | <2.5 | <2.5 | <2.5 | <2.5 |
| | C + S | | | | | | |
| | (2500 ppm) | | | | | | |
| S12 | PROSIDIUM | + | <2.5 | <2.5 | <2.5 | <2.5 | <2.5 |
| | C + S | | | | | | |
| | (5000 ppm) | | | | | | |

ASFV = African swine fever virus;
SC RME = Sal CURB RM E Liquid, a formaldehyde-based product;
PROSIDIUM C + S = PROSIDIUM Concentrate containing PPA 16% and S is phosphate buffer applied at 9:1 ratio;
PPA = 17% PPA;
− = absence;
+ = present.

### Discussion

The data demonstrates the strong antiviral effect of PPA and PROSIDIUM C+S in solution. Although all three products were able to reduce ASFV to an undetectable level, the effects of PPA and PROSIDIUM C+S were demonstrated to be faster than a formaldehyde-based product (Sal CURB RME Liquid). PROSIDIUM C+S, which contains 16% PPA, can reduce ASFV of a minimum of 4 logs to an undetectable level within approximately 10 minutes at a concentration as low as 200 ppm. Further study in vitro feed showed that both products, PPA mixed with phosphate buffer and a formaldehyde-based product (Sal CURB RME Liquid) could reduce ASF-contaminated feed to below the detection limit within 1 hour after application.

### Example 5: Efficacy of PPA Compositions on High Pathogenic Avian Influenza (Type A) Viruses Inactivation

### Materials and Methods

Cell culture and virus. Madin-Darby Canine Kidney (MDCK) and Avian influenza (AIV) H5N1 were used for in this study. MDCK was cultured in Dulbecco's Modified Eagle Medium (DMEM) supplemented with 5% fetal bovine serum (FBS) and 1% antibiotics at 37° C. with 5% carbon dioxide (CO₂). Viral titer of AIV was at ca. 7 log (TCID₅₀/mL).

Treatments. The study was conducted in solution at a final contamination level of ca. 6 log (TCID₅₀/mL) with treatments and time points as tabulated in Table 9. PROSIDIUM C+S was applied with PROSIDIUM Concentrate containing PPA 16% and PROSIDIUM S (a buffer to temporally stabilize peroxyacid and reduce the aggressiveness of the PPA to feed mill equipment) at a 9:1 ratio of the stated concentrations.

TABLE 9
Treatments and assay conditions

| | | Concentration | AIV | Duration | |
|---|---|---|---|---|---|
| Code | Treatment | (ppm) | TCID₅₀/mL | (h) | Remarks |
| S1 | Sal CURB RME | <5000 | — | — | Cytotoxicity test |
| | Liquid SCRME | | | | of disinfectant |
| S2 | PROSIDIUM | <5000 | — | — | (2-fold serial |
| | C + S | | | | dilution) |
| S3 | PPA | <5000 | — | — | |
| S4 | Water | — | — | 0, 0.5, 1, 3, 6 | Negative control |
| S5 | Water | — | 10^{6.0} | 0, 0.5, 1, 3, 6 | Positive control |
| S6 | Sal CURB RME | 1000 | 10^{6.0} | 0, 0.5, 1, 3, 6 | — |
| | Liquid SCRME | | | | |
| S7 | PPA | 1000 | 10^{6.0} | 0, 0.5, 1, 3, 6 | — |
| S8 | PROSIDIUM | 200 | 10^{6.0} | 0, 0.5, 1, 3, 6 | — |
| | C + S | | | | |
| S9 | PROSIDIUM | 500 | 10^{6.0} | 0, 0.5, 1, 3, 6 | — |
| | C + S | | | | |
| S10 | PROSIDIUM | 1000 | 10^{6.0} | 0, 0.5, 1, 3, 6 | — |
| | C + S | | | | |
| S11 | PROSIDIUM | 2500 | 10^{6.0} | 0,0.5, 1, 3, 6 | — |
| | C + S | | | | |
| S12 | PROSIDIUM | 5000 | 10^{6.0} | 0, 0.5, 1, 3, 6 | — |
| | C + S | | | | |

50% tissue culture infectious dose (TCID₅₀) assay. Treatments were prepared at 1.11 times higher of the stated concentration to account for the dilution upon the addition of virus. The prepared treatment solution was added in 9 parts to 1 part of virus solution. Briefly, 900 μL of the prepared treatment solution (at 1.11 times higher) is added to 100 μL of virus solution. The mixture was vortexed to mix well. Subsequently, the treated virus solution was left to stand until the time points stated in Table 9. At each time interval, 250 μL of 0.1 M sodium thiosulphate is added to 1000 μL of the treated virus solution, mix well and leave to stand for 10 minutes for neutralization. After 10 minutes, the solution was serial diluted ten-fold with DMEM only for inoculation into 96-well plates already confluent with MDCK cells. 100 μL of the solution at each dilution was inoculated into four wells and incubated for one hour at 37° C. with 5% CO₂. After incubation for one hour, the inoculated solution in each well was removed and replaced with 200 μL of DMEM supplemented with 1% FBS and 1% antibiotics. Cytopathic effect was visually observed after four days of incubation at 37° C. with 5% CO₂, with TCID₅₀ calculated using Spearman-Karber method.

Cytotoxicity assay. Treatment was carried out the same way as TCID₅₀ assay. However, instead of virus, 900 μL of treatment solution was added to 100 μL sterile media (to which the virus is stored in) instead of to the virus solution. Subsequently, the mixture was serial diluted two-fold and added to 96-well plate already confluent with MDCK cells. Cytotoxic concentration was determined by visually observing the cells using an inverted microscope after 48 hours of incubation at 37° C. with 5% CO₂.

Cytotoxicity. The results showed that the maximum non-toxic concentration of Sal CURB RME Liquid (SC RME; a formaldehyde-based product) is the lowest at 156.25 ppm (Table 9). On the other hand, maximum non-toxic concentration of PROSIDIUM C+S and PPA is at 625.00 and 312.50 ppm. This indicated that Sal CURB RME Liquid is more toxic than PROSIDIUM C+S and PPA to MDCK cells.

TABLE 10
The maximum non-toxic concentration of treatments in ppm.

| | | Maximum non-toxic |
|---|---|---|
| Code | Sample | concentration, ppm |
| S1 | SC RME | 156.25 |
| S2 | PROSIDIUM C + S | 625.00 |
| S3 | PPA | 312.50 |

SC RME = Sal CURB RM E, a formaldehyde-based product; PROSIDIUM C (containing 16% PPA) + S (as a buffer) = PROSIDIUM C and S applied at 9:1 ratio; PPA = 17% PPA.

50% tissue culture infectious dose (TCID₅₀) assay. At timepoint 0, the neutralizing solution (i.e., sodium thiosulphate) was added to the treated virus solution within 10-15 minutes. The results showed that PPA and PROSIDIUM C+S can reduce AIV of a minimum of 4 logs within 10-15 minutes of treatment at a concentration as low as 1000 and 200 ppm respectively. On the other hand, the effect of Sal CURB RME Liquid is slower where it can only reduce up to 1.25 log (TCID₅₀/mL) of AIV after 10-15 minutes of treatment. Similar to PPA and PROSIDIUM C+S, Sal CURB RME Liquid can also reduce AIV to an undetectable level after six post-treatment hours.

TABLE 11
In vitro virucidal efficacy of various treatments/products
against high pathogenic avian influenza virus

| | | | AIV titer after treatment | | | | |
|---|---|---|---|---|---|---|---|
| | | | (log TCID₅₀/mL) | | | | |
| | | | Duration (h) | | | | |
| Code | Sample | Virus | 0 | 0.5 | 1.0 | 3.0 | 6.0 |
| S4 | Water | − | <1.5 | <1.5 | <1.5 | <1.5 | <1.5 |
| S5 | Water | + | 5.75 | 6.00 | 6.00 | 6.00 | 5.75 |
| S6 | SC RME | + | 4.50 | 4.00 | 4.00 | 2.50 | <2.5 |
| | (1000 ppm) | | | | | | |
| S7 | PPA | + | <2.5 | <2.5 | <2.5 | <2.5 | <2.5 |
| | (1000 ppm) | | | | | | |
| S8 | PROSIDIUM | + | <1.5 | <1.5 | <1.5 | <1.5 | <1.5 |
| | C + S | | | | | | |
| | (200 ppm) | | | | | | |
| S9 | PROSIDIUM | + | <2.5 | <2.5 | <2.5 | <2.5 | <2.5 |
| | C + S | | | | | | |
| | (500 ppm) | | | | | | |
| S10 | PROSIDIUM | + | <2.5 | <2.5 | <2.5 | <2.5 | <2.5 |
| | C + S | | | | | | |
| | (1000 ppm) | | | | | | |
| S11 | PROSIDIUM | + | <2.5 | <2.5 | <2.5 | <2.5 | <2.5 |
| | C + S | | | | | | |
| | (2500 ppm) | | | | | | |
| S12 | PROSIDIUM | + | <2.5 | <2.5 | <2.5 | <2.5 | <2.5 |
| | C + S | | | | | | |
| | (5000 ppm) | | | | | | |

AIV = Avian influenza (H5N1);
SC RME = Sal CURB RM E Liquid;
PROSIDIUM C + S = PROSIDIUM C (containing 16% PPA) and S applied at 9:1 ratio;
PPA = 17% PPA;
− = absence;
+ = present.

TABLE 12
In vitro virucidal efficacy of various treatments/products against
high pathogenic avian influenza virus-contaminated feed materials.

| | Treatment group | AIV titer after treatment with disinfectant; |
|---|---|---|
| Entry | Sample + Feed | incubation 1 hour (log10 TCID50/ml) |
| 1. | Water | 0 |
| 2. | Avian influenza virus | 6.75 |
| | stock (not in feed) | |
| 3. | AIV + Water in feed | 3.75 |
| 3. | SCRME (1000 PPM) | 3.50 |
| 4. | PPA (1000 PPM) | 2.75 |

AIV = Avian influenza (H5N1); SC RME = Sal CURB RM E Liquid; PPA = 17% PPA + buffer

### Discussion

PPA and PROSIDIUM C+S had a strong virucidal effect in solution. Although all three products could reduce AIV to an undetectable level, the virucidal effect of PPA and PROSIDIUM C+S was demonstrated to be faster than a formaldehyde-based product (Sal CURB RME Liquid). Furthermore, PROSIDIUM C+S can inactivate/reduce the virus titer to an undetectable level within 10-15 minutes at a concentration as low as 200 ppm. Further study in vitro showed that PPA mixed with phosphate buffer could reduce AIV contaminated feed within 1 hour after application. In contrast, the formaldehyde-based product doesn't show similar efficacy on AIV reduction at the same dose application and treatment time.

### Example 6: Antiviral Effect of PPA Against Porcine Epidemic Diarrhea Virus (PEDV) and its Inactivation Mechanism

### Materials and Methods

Cell culture and virus propagation. Following the method described by Thomas et al. (2015) with some modifications, Vero 81 cells were cultured using Eagle's minimum essential medium (EMEM; Gibco) supplemented with 10% fetal bovine serum (FBS, Cytiva) and 1% penicillin-streptomycin (Gibco) at 37° C. with 5% CO₂²⁰. Thomas et al. (2015), Effect of Porcine Epidemic Diarrhea Virus Infectious Doses on Infection Outcomes in Naïve Conventional Neonatal and Weaned Pigs. PloS one, 10 (10), e0139266.

PEDV (USA/NC49469/2013 strain; procured from Iowa State University was propagated in Vero 81 cells using EMEM supplemented with 0.3% tryptose phosphate broth (Sigma) and 10 ug/mL trypsin (Gibco) at 37° C. with 5% CO₂ for two days. After three freeze-thaw cycles, the virus lysate was collected and stored at −80° C.

50% Tissue culture infectious dose (TCID₅₀) assay. A 100 μL of PEDV (ca. 10⁴ TCID₅₀/mL) was treated with 900 μL of treatment solution, prepared in phosphate-buffered saline, for one hour at room temperature. The treated virus samples (200 μL) were inoculated into Vero 81 cells in 96-well culture plates and incubated for three days at 37° C. with 5% CO₂. To determine the effect of feed matrices on the virucidal effect of the treatments, the comparison was also carried out in feed extract, where treatments were prepared using feed extract instead of PBS. The feed extract was prepared by weighing 50 g of feed into 100 mL of PBS and stirring at room temperature for 10 minutes. To remove the feed particles, the collected supernatant was filtered through a 0.2 μm PES membrane (Thermo Fischer Scientific). PPA and Sal CURB LF Liquid were tested. After one hour, the treatments were neutralized with 667 μL of 0.1 M sodium thiosulphate (Sigma-Aldrich). The mixture was mixed well and left to stand for 10 minutes. The cytopathic effect was determined by observing visible cell clumping and broken monolayers. The 50% tissue culture infectious dose (TCID₅₀) was calculated using the Reed-Muench method.

Treatment for inactivation mechanism studies. The virus was treated for one hour at room temperature by adding 100 μL of PEDV (at ca. 10⁵ TCID₅₀/mL) to 900 μL of treatment solution, prepared in phosphate-buffered saline (PBS). Control is prepared by adding 900 μL of PBS to 100 μL of PEDV. After one hour, the treatment is neutralized with 667 μL of 0.1 M sodium thiosulphate for treatments using 500 and 2500 ppm of PPA and 0.5 M of sodium thiosulphate for 12500 ppm of PPA treatment for 10 minutes. Subsequently, 2500 ppm of PPA treatment is further neutralized with 1 μL of 5 M sodium hydroxide, whereas 12500 ppm of PPA treatment is neutralized with 1 μL of 10 M sodium hydroxide. To ensure all samples have the same dilution factor, 1 μL of sterile water was added to treatment with 500 ppm PPA and the control. The samples were subsequently evaluated for RNA and viral envelope damage.

Viral RNA damage. Viral RNA was extracted by using Genesig Easy RNA extraction kit (Primerdesign Ltd). The RNA was extracted according to the supplier's instructions, using 200 μL of treated viral samples and 100 μL of elution buffer. Five μL of the extracted RNA samples were added to 15 μL of the reaction mixture prepared using the Luna® Universal One-Step Reaction RT-qPCR kit (New England Biolabs) and analyzed using RT-qPCR with protocol and primer sequences detailed in Tables 13 and 14.

TABLE 13
RT-qPCR protocol

| | Cycle | Temperature | Duration |
|---|---|---|---|
| Step | number | (° C.) | (s) |
| Reverse transcription | 1 | 55 | 600 |
| Enzyme activation | 1 | 95 | 60 |
| Denaturation & data | 45 | 95 | 10 |
| collection | | 60 | 30 |
| Cooling | 1 | 40 | 30 |

TABLE 14
Primer sequences used for PEDV

| Primer | Sequence |
|---|---|
| Forward | 5′-AGT GCT TCA GAG GCT GAT TAC-3′ |
| Reverse | 5′-CAC AGT GGG TGG TGT GTA TAA-3′ |

Viral structural damage. One μL of RNase A was added to 200 μL of treated viral samples. The mixture was left to stand at room temperature for two hours. Subsequently, 20 μL of proteinase K (Primerdesign Ltd) was added to the mixture and incubated at 37° C. for 30 minutes. After incubation, RNA was extracted using the Genesig Easy RNA extraction kit. The quantification cycle was determined by RT-qPCR.

Statistical analysis. Statistical analyses were performed using Statsgraphics Centurion 18 software. One-way ANOVA was conducted using the Tukey test as a post hoc test. Results were considered significantly different at p<0.05.

### Results

Antiviral effect of PPA. PPA is effective against PEDV at a concentration higher than 20 ppm. It could reduce PEDV to an undetectable level (<1.7 log (TCID₅₀/mL)), indicating a reduction of at least 1.2 log (TCID₅₀/mL) of PEDV within one hour of treatment time in phosphate buffer saline (PBS) (FIG. 13A). A higher effective dose of PPA was required when in feed extract. Although the results have shown that feed matrices could affect the effective treatment concentration of PPA, the virucidal effect of PPA at 20 ppm is comparable to that of Sal CURB LF Liquid at 500 ppm (FIG. 13B). These results, therefore, indicated that PPA is potentially five times more potent towards PEDV than the formaldehyde-based Sal CURB LF Liquid, which is commonly used as a mitigant for PEDV in feed extract.

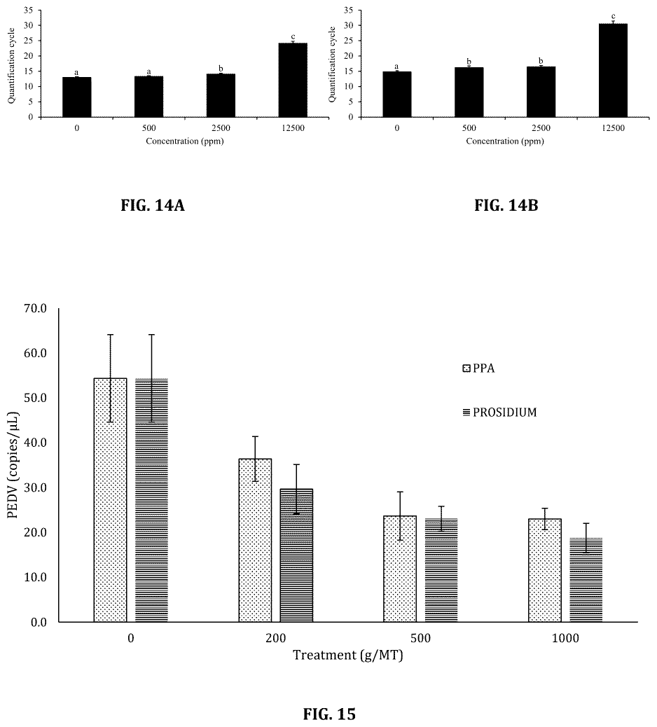

Viral RNA damage. A higher quantification cycle (Cq) value will indicate a lower amount of RNA and, therefore, a greater extent of RNA damage. The quantification cycle (Cq) observed at 500 ppm (Cq=13.1) was comparable to that of the control (Cq=13.4; FIG. 14A). However, as the concentration was increased to 2500 and 12500 ppm, significant RNA damage was observed compared to the control. This indicated that no significant RNA damage was observed at 500 ppm; however, PPA damaged RNA at a higher concentration of 2500 and 12500 ppm.

Viral structural damage. The effect of the PPA on the viral structure was evaluated using RNase A. The addition of RNase led to a significantly higher Cq value observed with PPA at 500 ppm (Cq=16.3) compared to the control (Cq=14.9; FIG. 14B), indicating a lower amount of RNA was present.

### Example 7. Efficacy of PPA and PROSIDIUM Against PEDV-Contaminated Feed

### Materials and Methods

Swine Feed. A commercial swine feed was obtained from Des Moines Feed Swine Diet (2024-09-20) ground and passed through 12 mesh sieve (2025-01-10). PED Virus-NC49469-2013 P8 PEDV VERO 27JAN21 HY (Found to be ~3×10⁶ copies/μL) was obtained from Iowa State University Veterinary Diagnostic Laboratory. A quantity of 6 mL virus was inoculated to 54 g of feed in 3×2 mL doses, with 1 minute of mixing in between. After 50 g of this feed was inoculated into 550 g of feed and mixed for 5 minutes.

Feed samples: 25 g samples (±0.03 g) were prepared for: Uninoculated Control, Untreated Control, PPA (16-17% PPA content)+Phosphate Buffer (200 g/MT, 500 g/MT, 1000 g/MT, 2500 g/MT, 5000 g/MT), PROSIDIUM (16-17% PPA)+Phosphate Buffer (200 g/MT, 500 g/MT and 1000 g/MT).

TABLE 15
Treatment Group Composition Details

| Group | Feed State | Composition of Treatment |
|---|---|---|
| 1 | Uninoculated-feed | ASTM Distilled Water LabChem Cat |
| | with no virus | LC267404 Lot# 8409442 |
| 2 | Untreated feed + virus | ASTM Distilled Water LabChem Cat |
| | (control) | LC267404 Lot# 8409442 |
| 3 | Feed + virus + treatment | PPA (16-17%) |
| | | hydrogen peroxide (8%) |
| | | sodium phosphate buffer |
| 4 | Feed + virus + treatment | PROSIDIUM |

All treatments were prepared at 125 mg/mL as stocks. Afterward, the stock of each solution was weighed out at 6.25 g to a 50 mL volumetric flask and brought to volume with water. Treatments were further diluted with water to 62.5 mg/mL, 25 mg/mL, 12.5 mg/mL, and 5 mg/mL The mentioned treatments were applied to the appropriate 25 g sample (contaminated feed and control feed) in 2×0.5 mL doses with a Hamilton 0.5 ml syringe (Hamilton 80800 750N 500 μL 22 gauge 2″) with 1 minute of mixing after each application. All 25 g feed samples were stored at 4° C. overnight. The following day, triplicate 5 g (+0.07 g) feed samples were taken from each treatment and combined with 25 mL of phosphate-buffered saline (Cytiva cat #SH30255.01, lot #AK30805382) in a 50 mL conical tube, mixed thoroughly for 1 minute, and then stored at 4° C. overnight.

RNA Purification: From the 5 g feed/25 mL PBS samples, 1 mL was taken and spun down at 15,000×g for 5 minutes. From this sample, 200 μL was taken, and the RNA was extracted using Genesig® Easy DNA/RNA kit (Cat #ZgenesigEASY-EK) containing 4 μL of internal extraction control, eluting to 200 μL.

Step 1:200 μL sample combined with 20 μL proteinase K, 5 μL carrier DNA, 200 μL lysis buffer. The sample was mixed and incubated at room temperature for 15 minutes.

Step 2: Mix 500 μL magnetic beads/binding buffer and wait 5 minutes. The samples were placed in the magnetic separator and waited 2 minutes before removing the supernatant.

Step 3: The tube was removed from the magnetic separator, and 500 μL of wash buffer was added, resuspending the beads fully by pipetting up and down. After 30 seconds, samples were placed in the magnetic separator. After 2 minutes, the supernatant was removed.

Step 4: The Tube was removed from the magnetic separator, and 500 μL of wash buffer 2 was added, resuspending the beads fully by pipetting up and down. After 30 seconds, samples were placed in the magnetic separator. After 2 minutes, the supernatant was removed from the tube.

Step 5: The tube was removed from the magnetic separator, and 500 μL of 80% ethanol was added, resuspending the beads fully by pipetting up and down. After 30 seconds, samples were placed in the magnetic separator. After 2 minutes, the supernatant was then removed.

Step 6: The magnetic bead pellet was allowed to dry for 10 minutes at room temperature with the lid open

Step 7: The Tube was removed from the magnetic separator, and 200 μL of Elution Buffer and the beads were resuspended fully by pipetting up and down. After waiting 30 seconds, samples were placed in the magnetic separator, and the supernatant containing the RNA was transferred to a 0.5 mL tube for storage (−80 C) and further analyze or use.

RNA Quantitation: Quantitation of RNA samples (5 L) was run in quadruplicate using the Genesig® Advanced kit for PEDV on Roche Lightcycler 480. RT-qPCR reactions were assembled on ice:

5 μL extracted RNA, 10 μL oasig OneStep 2×RT-qPCR Master Mix, 1 μL PEDV primer/probe mix, 1 μL Internal extraction control primer/probe mix, 3 μL RNase/DNase free water. Negative controls replaced RNA with water. A standard curve was prepared with a dilution series of the Positive Control (2×10⁵ copies/L), diluting 10 μL of the positive control with 90 μL of template preparation buffer. This allowed for a 6-point standard curve.
Tube 1-2×10⁵ copies/μL
Tube 2-2×10⁴ copies/μL
Tube 3-2×10³ copies/μL
Tube 4-2×10² copies/μL
Tube 5-2×10¹ copies/μL
Tube 6-2×10⁰ copies/μL

Duplicates of each standard and negative control were run on each plate.

Plates were Run Combining Similar Treatment Rates:
Plate 1—Uninoculated control, 200 g/MT (PPA with buffer, PROSIDIUM with buffer)
Plate 2—Untreated control, 500 g/MT (PPA with buffer, PROSIDIUM with buffer)
Plate 3—1000 g/MT (PPA with buffer, PROSIDIUM with buffer)

### Results

FIG. 15 shows the antiviral effect of PPA (16-17%) with phosphate buffer and PROSIDIUM (containing 16-17% PPA) with buffer in animal feed. PPA is effective against PEDV-contaminated feed (Swine feed) at a concentration as low as 200 g/MT. The treatment reduced the presence of PEDV in the feed by more than 50% of the initial level of PEDV contamination in feed within a short period of treatment time, roughly 10-15 minutes, in phosphate-buffered saline (PBS) (FIG. 15).

### Discussion

To the inventors' knowledge, the application of PPA in feed and against PEDV was not previously reported. Furthermore, the in-depth study on its mode of action is limited. This work investigated the antiviral effect of PPA against PEDV and its underlying mechanisms. In addition, the effectiveness of PPA was compared to a formaldehyde-based product, Sal CURB LF Liquid. These results demonstrated that the PPA exhibited strong virucidal activity in both phosphate buffer and feed extract (FIG. 13). However, it was found that a higher effective dosage was required when tested in feed extract. Using the feed extract (in phosphate buffer), 100 ppm of PPA was required to reduce PEDV to an undetectable level (<1.7 log (TCID₅₀/mL)) (FIG. 13B). The high reactivity of PPA could have accounted for this difference, where PPA reacted with the organic matter in the feed extract. In contrast to a report by Phillips et al (2022)¹², no significant viral reduction was observed with a formaldehyde-based product (Sal CURB LF Liquid). The results indicated that PPA is potentially five times more potent towards PEDV in feed extract than Sal CURB LF (FIG. 13B). While a strong virucidal effect was observed at 20-100 ppm, the mode of action study on viral RNA damage used concentrations ranging from 500 to 12500 ppm to better observe the dose-dependent effect. One hypothesis is that PPA damages the amino acids and glycerophospholipids in the viral structure, preventing virus mediation to host interaction and, therefore, the loss of viral infectivity. The damage of the viral structure could allow for the better penetration of PPA into the intracellular space, thus damaging the viral envelope and RNA, as demonstrated by RT-qPCR.

The effect of PPA on the viral structure was evaluated by adding RNase A to the treated viruses. With structural damage, RNase A can enter the virions, thus digesting the RNA and leading to a higher quantification cycle. A higher quantification cycle (Cq) value will indicate a lower amount of RNA and, therefore, a greater extent of RNA damage. Adding RNase A to the treated virus resulted in a statistically significant higher quantification cycle value at 500 ppm (FIG. 14B). This suggested that PPA damaged the viral structure, allowing RNase A to access the interior space and digest the RNA. This thus confirmed that PPA inactivated PEDV via structural damage.

In conclusion, these results show that PPA demonstrated noteworthy potential as a broad-spectrum antiviral agent and furthermore identified structural damage as the main inactivating mechanism for PEDV.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

It should be further appreciated that minor dosage and formulation modifications of the composition and the ranges expressed herein may be made and still come within the scope and spirit of the present invention.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

1-17. (canceled)

18. A method of controlling growth or spread of a virus in animal feed or water, comprising applying to the animal feed or water a composition comprising one or more peroxyacid; wherein the composition contains an effective amount of the one or more peroxyacid to reduce the viral load of the animal feed or water compared to an untreated animal feed or water.

19. The method of claim 18, wherein the virus is selected from the group consisting of African Swine Fever virus, porcine epidemic diarrhea virus, foot-and-mouth disease virus, bovine viral diarrhea virus, Avian Influenza virus, influenza A virus of swine, bovine herpesvirus type 1, porcine reproductive and respiratory syndrome virus, and avian paramyxovirus Newcastle disease virus.

20. The method of claim 18, wherein the one or more peroxyacid is peroxypropionic acid.

21. (canceled)

22. The method of claim 18, wherein the composition further comprises an acid selected from the group consisting of propionic acid, acetic acid, citric acid, and blends thereof.

23. The method of claim 18, wherein the composition further comprises a peroxide.

24. The method of claim 18, wherein the composition is a wet feed additive or a dry feed additive.

25. (canceled)

26. The method of claim 18, wherein the composition does not contain formaldehyde.

27. The method of claim 18, wherein the composition does not contain formic acid.

28. The method of claim 18, wherein the composition further comprises at least one of a surfactant or an emulsifier.

29-36. (canceled)

37. A method of reducing an outbreak or spread of a viral disease in livestock comprising adding to a livestock feed or water supply, or applying to a surface that comes into contact with the livestock feed or water supply, an effective amount of a composition comprising one or more peroxyacids.

38. The method of claim 37 wherein the composition comprises a buffered peroxyacid.

39. The method of claim 37 wherein the one or more peroxyacid includes peroxypropionic acid.

40. The method of claim 39 wherein the composition contains an effective amount of peroxypropionic acid to control the growth of a virus that causes the viral disease, and wherein the virus is selected from the group consisting of African swine fever virus, porcine epidemic diarrhea virus, and avian influenza virus.

41-42. (canceled)

43. The method of claim 37, wherein the composition does not contain formaldehyde.

44-45. (canceled)

46. An anti-viral kit for treating animal feed comprising:
a first component containing a peroxyacid concentrate;
a second component containing a phosphate buffer;
instructions for mixing the first component and the second component in order to form a composition that contains the peroxyacid in an amount effective to inactivate a virus in the animal feed.

47. The kit of claim 46, wherein the first component comprises peroxypropionic acid.

48. The kit of claim 46, wherein the virus is selected from the group consisting of African Swine Fever virus, porcine epidemic diarrhea virus, foot-and-mouth disease virus, bovine viral diarrhea virus, Avian Influenza virus, influenza A virus of swine, bovine herpesvirus type 1, porcine reproductive and respiratory syndrome virus, and avian paramyxovirus Newcastle disease virus.

49. The method of claim 37, wherein the composition further comprises an acid selected from the group consisting of propionic acid, acetic acid, citric acid, and blends thereof.

50. The method of claim 37, wherein the viral disease is caused by a virus selected from the group consisting of African Swine Fever virus, porcine epidemic diarrhea virus, foot-and-mouth disease virus, bovine viral diarrhea virus, Avian Influenza virus, influenza A virus of swine, bovine herpesvirus type 1, porcine reproductive and respiratory syndrome virus, and avian paramyxovirus Newcastle disease virus.

51. The method of claim 37, wherein the composition reduces a viral load associated with the viral disease to an undetectable level.
